(19) United States
(12) Patent Application Publication
(10) Pub. No.: US 2007/0092496 A1
(43) Pub. Date: Apr. 26, 2007

(54) METHOD OF DELIVERING CELLS TO THE SKIN

(75) Inventors: Ying Zheng, Wayne, PA (US); Xiaobing Du, Philadelphia, PA (US); Kenneth Justin Washenik, Beverly Hills, CA (US); Kurt Stricker Stenn, Princeton, NJ (US)

Correspondence Address: MICHAEL BEST & FRIEDRICH, LLP, ONE SOUTH PINCKNEY STREET, P O BOX 1806, MADISON, WI, 53701, US

(73) Assignee: Aderans Research Institute, Inc., Beverly Hills, CA (US)

(21) Appl. No.: 11/550,007

(22) Filed: Oct. 17, 2006

Related U.S. Application Data
(60) Provisional application No. 60/727,588, filed on Oct. 17, 2005.

(51) Int. Cl. A61K 35/32

(52) U.S. Cl. 424/93.7

(57) ABSTRACT

The present invention is a method of forming egressing hair shafts comprising making a wound in the skin and placing hair follicle progenitor cells on the wound, wherein an egressing hair shaft is formed from the hair follicle progenitor cells. The present invention is also a method of forming new skin comprising making a wound in the skin and placing skin forming cells on the wound, wherein new skin is formed from the skin forming cells.

### CROSS-REFERENCE TO RELATED APPLICATIONS

This application claims the benefit of U.S. Provisional Application Ser. No. 60/727,588, filed Oct. 17, 2005, which is incorporated by reference herein.

### STATEMENT REGARDING FEDERALLY SPONSORED RESEARCH OR DEVELOPMENT

Not applicable.

### BACKGROUND OF THE INVENTION

Developing appropriate cell delivery techniques is important for many applications. Not only must the cells be delivered to the appropriate part of the subject, but the morphology of the cells must be maintained. The morphology of cells can be important in many contexts. Cell morphology indicates the status of the cells, both in terms of the health of the cells and in terms of the differentiation state of the cell. Changes in cell shape, or morphogenesis, are central to cell function, development and disease. Physiological processes in cells are often accompanied by changes in cellular morphology. Examples include changes in the intracellular location, arrangement and structure of cellular constituents, such as organelles, macromolecular clusters or the cytoskeleton, changes in the morphology of the entire cell, such as its shape and area, changes in the spacing and proximity between cells, and properties of multi-cellular colonies such as its shape, size and cell locations.

### SUMMARY OF THE INVENTION

In one embodiment, the present invention is a method of forming egressing hair shafts comprising making a wound in the skin and placing hair follicle progenitor cells on the wound, wherein an egressing hair shaft is formed from the hair follicle progenitor cells. The present invention is also a method of forming new skin comprising making a wound in the skin and placing skin forming cells on the wound, wherein new skin is formed from the skin forming cells.

### BRIEF DESCRIPTION OF THE FIGURES

FIG. 1 shows a grid of superficial cuts on the surface of the skin of a nude mouse and hair out-growth (inset) observed at two weeks post-administration with black newborn mouse skin cells.

FIG. 2 shows a scalpel blade being used to make a superficial stab wound on the surface of the skin of a nude mouse and hair out-growth (inset) observed at two weeks post-administration of black newborn mouse skin cells into the wound.

### DETAILED DESCRIPTION OF THE INVENTION

It has been discovered that cells can be delivered into the superficial skin without disturbing the inherent characteristics of the cells by making a wound in an area where the cells are desired and placing the cells on the wound. It is expected that the cells would orientate themselves appropriately to form the desired structure upon delivery with the method of the present invention. Suitably, the cells can be placed on the surface of the skin on top of the wound or in the wound itself.

The cell delivery method of the present invention can be used to form egressing hair shafts. The cell delivery method of the present invention may also be used to form new skin to treat, repair or improve conditions including, but not limited to, skin ulcers, diabetic foot ulcers, bed sores, burn wounds, microbial infections, and scars such as those resulting from surgery, acne, or illnesses such as chicken pox. Delivery of appropriate cells by the methods of the present invention could also be used to form sweat glands, nail, eyebrow, eyelash and other hairs. The method of the present invention could also be used to form the dermis or epidermis using genetically altered autologous or allogenic cells. Suitably, the cell delivery method of the present invention may be used to form both the dermal and epidermal layers of the skin. Alternatively, either the dermal or the epidermal layers may be formed by the method of the present invention.

The wound may be formed by any technique that disrupts the outer surface of the area in which the cells are desired such as stabbing, cutting or scratching the area with a sharp instrument, including, but not limited to, a scalpel blade or a needle. The wound may also be formed by abrading the area with, e.g. needles such as microneedles or grooved needles.

For certain embodiments, the sharp instrument may be modified to form a wound with a measured depth. For example, the wound may be at least about 10 μm in depth, at least about 200 μm in depth, no more than about 500 μm in depth, or no more than about 250 μm in depth. The wound may be from about 10 μm to about 500 μm in depth. The wound may be unlimited in maximal length. In certain embodiments of the invention, the depth of the wound may be adjusted to deliver the cells into the subepidermis, the papillary dermis, the upper reticular dermis, or the same levels of the nail bed of the acral skin (palmer-plantar skin). Suitably, the wound is shallow enough that the subject does not bleed. The wound suitably heals without forming a scar.

After the cells are placed on the wound, the wound may be covered with a bandage or dressing; for example, a non-adhering dressing, or a transparent plastic dressing such as Tegaderm® (3M, St. Paul, Min.) or a gel-based burn dressing. Petroleum jelly or the like may also be applied to the wound. The dressing may be left in place for about 3 to about 7 days. Suitably, the dressing may be substantially water-impermeable.

The cells may comprise dermal cells, epidermal cells, epidermal stem cells, basal cells, keratinocytes, fibroblasts or combinations thereof. The cells may be derived from follicular, eccrine or nail sources. Suitably, the cells are skin forming cells or hair follicle progenitor cells. For certain embodiments of the present invention, the ratio of epidermal to dermal cells is suitably about 10:1 to about 1:10. Suitably, the ratio may be about 1:1 to about 5:1.

The cells may suitably be provided in a suspension in a physiologically acceptable carrier, e.g., sterile saline solution. Additional components may also be added to the cell suspension. Suitable additional components include growth factors, nutrient molecules or stabilizing molecules.

The following examples are provided to assist in further understanding of the invention. The particular materials and methods employed are considered to be illustrative of the invention and are not meant to be limiting on the scope of the claims.

### EXAMPLES

### Example 1

### Hair Outgrowth from Follicle-Inductive Cells Administered to Superficial Cuts on the Surface of Mouse Skin

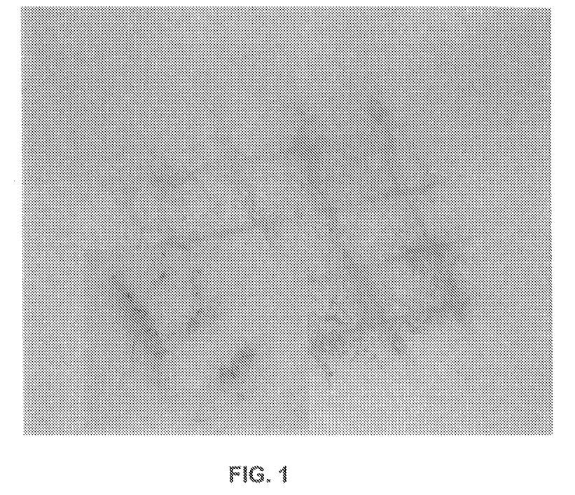

An athymic nude (nu/nu) mouse (Charles River, Inc.) was anesthetized and a grid of superficial cuts (FIG. 1) was made on the dorsal skin with the use of a number 11 scalpel blade. The cuts were shallow enough not to draw blood. A mixture of freshly isolated newborn black mouse skin cells comprising 100,000 epidermal cells and 200,000 dermal cells was prepared as follows. Truncal skin was removed from newborn mice and rinsed in Ca²⁺ and Mg²⁺ free PBS. The skin was laid flat in PBS containing Dispase (2.5 mg per ml, Invitrogen, Carslbad, Calif.) at 4° C. overnight or 37° C. for 2 hours. Inductive dermal and epidermal cells were isolated as described in Weinberg et al., J. Invest. Dermatol., 100:229-236 (1993), which is incorporated herein by reference. A suspension of the cells in 2 microliters of sterile buffered saline solution was delivered to the grid-of-cuts by pipette. The skin was gently pulled apart for a few seconds to allow the fluid to wick throughout the grid. A non-adherent, hydrophobic (petrolatum coated gauze) dressing was applied to the wound and the mouse was further bandaged with an elastic wrap to prevent removal of the dressing upon recovery from anesthesia. The dressing was removed after 10 days. After 2 weeks the growth of hair was observed (FIG. 1, inset) primarily within the grid pattern. The mouse was then euthanized and the skin gently removed. Observation of the underside of the skin revealed almost no ingrown hairs and the presence of follicle bulbs corresponding to the visible hair seen on the skin surface.

### Example 2

### Hair Outgrowth from Follicle-Inductive Cells Administered to Superficial Stab Wound on the Surface of Mouse Skin

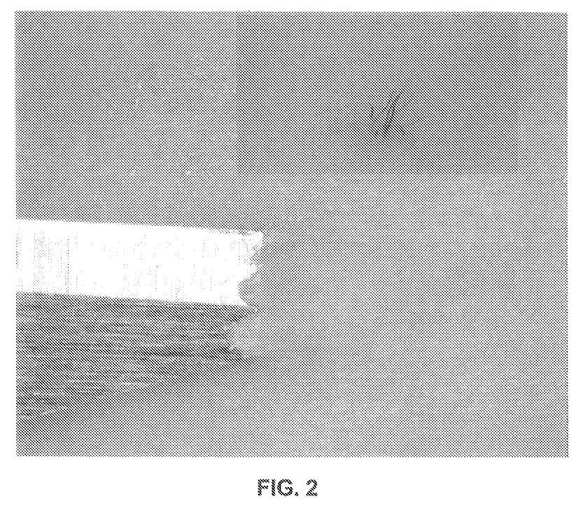

An athymic nude (nu/nu) mouse (Charles River, Inc.) was anesthetized and a stab wound was made in the skin by piercing with the tip of a number 11 scalpel blade held a low angle to the surface of the skin (FIG. 2). A mixture of freshly isolated newborn black mouse skin cells comprising 100,000 epidermal cells and 200,000 dermal cells was prepared as described above. A suspension of the cells in 2 microliters of sterile buffered saline solution was instilled into the cut by pipette. A non-adherent, hydrophobic (petrolatum coated gauze) dressing was applied to the wound and the mouse was further bandaged with an elastic wrap to prevent removal of the dressing upon recovery from anesthesia. The dressing was removed after 10 days. After 2 weeks the growth of hair was observed (FIG. 2, inset) precisely at the site of the stab incision. The mouse was then euthanized and the skin gently removed. Observation of the underside of the skin revealed almost no ingrown hairs and the presence of follicle bulbs corresponding to the visible hair seen on the skin surface.

### Example 3

### Use of Different Wound Dressing Materials

Examples 1 and 2 were repeated using different types of wound dressing materials as shown in Table 1.

TABLE 1
Superficial Delivery of Mouse Cells into Nude Mice

| | | # of Newborn Black | | | |
|---|---|---|---|---|---|
| Delivery | Type of | Mouse Skin Cells | Vol. | Number of | Outgrowth |
| Method | Dressing | Delivered | (μL) | Replicates | Rate |
| Example 1 | None | 1 × 10⁵ Epidermal mixed with | 2 | 8 | 0 |
| | | 2 × 10⁵ Dermal | | | |
| Example 1 | Non-adhering | 1 × 10⁵ Epidermal mixed with | 2 | 6 | 6 |
| | | 2 × 10⁵ Dermal | | | |
| Example 1 | Burn pad | 1 × 10⁵ Epidermal mixed with | 2 | 4 | 3 |
| | | 2 × 10⁵ Dermal | | | |
| Example 1 | Tegaderm ™ | 1 × 10⁵ Epidermal mixed with | 2 | 5 | 2 |
| | | 2 × 10⁵ Dermal | | | |
| Example 2 | None | 1 × 10⁵ Epidermal mixed with | 2 | 12 | 5 |
| | | 2 × 10⁵ Dermal | | | |
| Example 2 | Non-adhering | 1 × 10⁵ Epidermal mixed with | 2 | 16 | 9 |
| | | 2 × 10⁵ Dermal | | | |
| Example 2 | Burn pad | 1 × 10⁵ Epidermal mixed with | 2 | 5 | 5 |
| | | 2 × 10⁵ Dermal | | | |
| Example 2 | Tegaderm ™ | 1 × 10⁵ Epidermal mixed with | 2 | 5 | 2 |
| | | 2 × 10⁵ Dermal | | | |

### Example 4

### New Hair Growth in Bald Scalp by application of Autologous Cells into Superficial Incisions

Superficial wounds are created on a human subject's head with a surgical blade or scalpel, as grids, multiple crosses, parallel lines or similar such pattern to achieve the desired cosmetic effect for hair growth. Autologous human trichogenic dermal and epidermal cells (optionally mixed with hair follicle melanocytes if needed to restore hair pigmentation) in suspension are then spread on top of the superficial cuts with pipette tips or syringes and the wound is covered for 2-3 days to prevent the cells from drying out prior to being incorporated into the skin. Egressing hair follicles are then formed.

### Example 5

### New Hair Growth in Bald Scalp by application of Dermal Cells into Superficial Incisions

Wounds are created on a human subject's head with a surgical blade or scalpel, as grids, multiple crosses, parallel lines or similar such pattern to achieve the desired cosmetic effect for hair growth. Human trichogenic dermal cells alone (optionally mixed with hair follicle melanocytes if needed to restore hair pigmentation) in suspension are then spread on top of the superficial cuts with pipette tips or syringes and the wound is covered for 2-3 days to prevent the cells from drying out prior to being incorporated into the skin. Egressing hair follicles are then formed.

### Example 6

### Formation of New Skin to Treat a Subject with a Diabetic Foot Ulcer

A subject with a diabetic foot ulcer is anesthetized under local or general anesthesia and a series of superficial cuts is made in the ulcer. The cuts are shallow enough not to draw blood. A suspension of basal cells in 2 microliters of sterile buffered saline solution is delivered to the cuts by pipette. The skin is gently pulled apart for a few seconds to allow the fluid to wick throughout the grid. A non-adherent, hydrophobic (petrolatum coated gauze) dressing is applied to the wound. The dressing is removed after 10 days. New skin forms and the ulcer is healed.

### Example 7

### Formation of New Skin to Treat a Subject with Bed Sores

A subject with bed sores is anesthetized under local or general anesthesia and a series of superficial cuts is made in the bed sore. The cuts are shallow enough not to draw blood. A suspension of basal cells in 2 microliters of sterile buffered saline solution is delivered to the cuts by pipette. The skin is gently pulled apart for a few seconds to allow the fluid to wick throughout the grid. A non-adherent, hydrophobic (petrolatum coated gauze) dressing is applied to the wound. The dressing is removed after 10 days. New skin forms and the bed sore is healed.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. All publications, patents and patent applications are herein expressly incorporated by reference to the same extent as if each individual publication or patent application was specifically and individually indicated by reference. In case of conflict between the present disclosure and the incorporated patents, publications and references, the present disclosure should control.

It also is specifically understood that any numerical range recited herein includes all values from the lower value to the upper value, i.e., all possible combinations of numerical values between the lowest value and the highest value enumerated are to be considered to be expressly stated in this application. For example, if a concentration range is stated as 1% to 50%, it is intended that values such as 2% to 40%, 10% to 30%, or 1% to 3%, etc., are expressly enumerated in this specification. If a concentration range is “at least 5%, it is intended that all percentage values up to and including 100% are also expressly enumerated. These are only examples of what is specifically intended.

The invention has been described with reference to various specific embodiments and techniques. However, it should be understood that many variations and modifications may be made while remaining within the spirit and scope of the invention.

1. A method of forming egressing hair shafts comprising making a wound in the skin and placing hair follicle progenitor cells on the wound, wherein an egressing hair shaft is formed.

2. The method of claim 1, wherein the cells comprise dermal cells.

3. The method of claim 1, wherein the cells comprise epidermal cells.

4. The method of claim 1, wherein the cells are placed in subepidermal skin.

5. The method of claim 1, wherein the cells are placed in papillary dermal skin.

6. The method of claim 1, wherein the cells are placed in upper reticular dermal skin.

7. The method of claim 1, wherein the wound is at least about 10 μm in depth.

8. The method of claim 1, wherein the wound is at least about 200 μm in depth.

9. The method of claim 1, wherein the wound is no more than about 500 μm in depth.

10. The method of claim 1, wherein the wound is no more than about 250 μm in depth.

11. The method of claim 1, further comprising covering the wound containing hair follicle progenitor cells with a wound dressing.

12. A method of forming new skin comprising making a wound in an area in need of new skin and placing skin forming cells on the wound, wherein new skin is formed.

13. The method of claim 12, wherein the cells comprise basal cells.

14. The method of claim 12, wherein the cells are placed in subepidermal skin.

15. The method of claim 12, wherein the cells are placed in papillary dermal skin.

16. The method of claim 12, wherein the cells are placed in upper reticular dermal skin.

17. The method of claim 12, wherein the wound is at least about 10 μm in depth.

18. The method of claim 12, wherein the wound is at least about 200 μm in depth.

19. The method of claim 12, wherein the wound is no more than about 500 μm in depth.

20. The method of claim 12, wherein the wound is no more than about 250 μm in depth.

21. The method of claim 12, further comprising covering the wound containing the skin forming cells with a wound dressing.
